(19) United States
(12) Patent Application Publication
(10) Pub. No.: US 2026/0087427 A1
(43) Pub. Date: Mar. 26, 2026

(54) METHOD AND SYSTEM FOR MATCHING TASK RELATING TO CATTLE WITH ENTITY

(71) Applicant: SENOVATE AI CO., LTD., Bangkok (TH)

(72) Inventors: Davids MAKARARPONG, Bangkok (TH); Chaidate INCHAISRI, Nakhonpathom (TH); Piyanush TOOPTOMPONG, Patumthani (TH); Pongsanun KHAMTA, Amnatcharoen (TH); Apirak TUDSORN, Phetchabun (TH); Aekaluck LEKLERDSIRIWONG, NaKornPatom (TH)

(73) Assignee: SENOVATE AI CO., LTD., Bangkok (TH)

(21) Appl. No.: 19/111,220

(22) PCT Filed: Sep. 12, 2022
(86) PCT No.: PCT/TH2022/050006

§ 371 Date: Mar. 12, 2025

(51) Int. Cl. G06Q 10/0631

(52) U.S. Cl. CPC G06Q 10/06311

(57) ABSTRACT

The present invention discloses a method for matching a task relating to cattle with an entity, comprising the steps of: generating a query for the task relating to cattle; obtaining task details, wherein the task details include a task information and a task location; obtaining a plurality of candidate entities using the task information, wherein the candidate entities include a candidate location and a candidate rating; obtaining an environmental data responding to the task details; determining at least one selected entity using the steps of applying at least one weight factor to the task details, the candidate entities, and the environmental data and calculating a score using the task details, the candidate entities, and the environmental data; and responding to the query with the selected entity.

### FIELD OF INVENTION

The present invention relates to a method for matching a task relating to cattle with an entity and the system for achieving the same.

### BACKGROUND OF THE INVENTION

It is known in the arts that primitive means for detecting notable conditions of cattle, including estrus and sickness, may include a simple observation by human. Such method can lead to undetected conditions due to human errors, and more significantly, from lack of indicative symptoms related to the conditions, such as a silent heat, which is prevalent in certain environmental temperature and humidity. These undetected conditions can lead to missed opportunity for insemination and low conception rate of the cattle.

Conventionally, methods and systems are developed for monitoring and detecting the conditions of cattle. For instance, a system may record relevant data locally where the data must be obtained by cattle owners. The owners will then manually interpret and report any *notable conditions to veterinarian or animal care providers. Therefore, the system can be time-consuming and error-prone due to the possibility of misinterpretation by the cattle owners.

The methods and systems may further be developed to interpret the data to determine the conditions. However, the interpretation is conducted statically with a predetermined set of criteria, and thus, cannot adapt based on different cattle management and environmental factors.

Moreover, some conditions when detected may require certain specialties including specific care providers. Cattle owners can experience difficulties in finding such providers who offer appropriate services in their local area or they might find an inexperienced care provider in respect to the required tasks.

United States Patent Application Publication No. 2017/0193580 A1 discloses a system and method for animal owners to find animal care providers with desired characteristics. The method includes the process of weighting factors based on desired factors and may utilize a distance between the owner and the providers. The method and system do not explicitly incorporate the use of sensor or the determination of potential tasks.

Korean Patent Application Publication No. 10-2019-0065822 involves a medical care matching system between pet owners and veterinary care providers. The system matches the two by comparing the location information of the veterinary care provider terminal with respect to the location of the pet owners. The location information is used as the primary determinant and weigh factors are not explicitly disclosed. Moreover, the determination of potential tasks is not specifically described, similarly to the above-mentioned publication.

Concerning a detection and determination system, European Patent Application Publication No. EP 3660855 A1 describes the system configured to collect telemetric, geographic, and other sensory data and forward the data to a user device and a remote server where the data is analyzed for activity of the animal and any possible animal situations. The publication does not disclose means of matching and selecting care providers in response to the situations.

It should also be note that contextual data in relative to the location of cattle is not found to be utilized in conventional methods and systems for monitoring conditions and matching appropriate care providers.

Therefore, there is a need to develop a system and method that is more efficient in matching notable conditions with appropriate care providers in order to take necessary actions in response to the conditions as well as monitoring, determining, and notifying various conditions of cattle.

### SUMMARY OF THE INVENTION

It is an objective of the present invention to provide a method and system, which can accurately detect notable conditions of cattle in a timely manner and match appropriate entities, which can execute tasks corresponding to the conditions upon the cattle.

In order to achieve the above objective, an embodiment of the present invention provides a method for matching a task relating to cattle with an entity, comprising the steps of: generating a query for the task relating to cattle; obtaining task details, wherein the task details include a task information and a task location; obtaining a plurality of candidate entities using the task information, wherein the candidate entities include a candidate location and a candidate rating; obtaining an environmental data corresponding to the task details; determining at least one selected entity using the steps of applying at least one weight factor to the task details, the candidate entities, and the environmental data and calculating a score using the task details, the candidate entities, and the environmental data; and responding to the query with the selected entity.

According to the embodiment, the method is able to match the task with the entity appropriately using the weight factors of the data. Specifically, the weight factors can be adjusted individually based on the importance of its connected information. The adjustment can be conducted by artificial intelligence or machine learning in order to determine the selected entity relevant to and capable of executing the task, such as an entity certified or skilled in a specific field. Therefore, the method can dynamically connect an entity with suitable role and responsibility to the task.

Moreover, the method will take account the environmental data which can include any related data of the task location which is the location of cattle. Such data can be additional factors and may be applied its specific weight factor for determining the entity. Further, the environmental data may include biosurveillance related to the cattle location.

Secondly, the steps of the method are conducted in real time. As described, according to the embodiment, the steps of obtaining the task details, obtaining the candidate entities, and obtaining the environmental data are conducted simultaneously upon the step of generating the query for the task relating to cattle. Hence, the information will be up to date.

Additionally, the steps of obtaining the task details and obtaining the candidate entities are conducted simultaneously. Therefore, potential mismatches due to information change may be lessen which would help ensure that the selected entity is corresponding to the task.

For example, the method can re-evaluate the condition of cattle based on circumstances and change the task detail required for the cattle. With such embodiment, the selected entity will still match the task. As another example, the method can receive the updated environmental data, such as sudden occurrences of severe weather or outbreak, and is adapted to determine an appropriate entity based on such occurrences.

Thirdly, according to the embodiment, the task location and the candidate location are processed to determine a candidate distance, and wherein the task information, the candidate distance, the candidate rating, and the environmental data are applied the weight factors and calculated for the score to determine the selected entity.

Accordingly, the method will take into account the distances between potential candidates and the task location and will be able make appropriate suggestions based on transportation costs for the potential candidates.

In combination with another embodiment, the method can obtain the candidate locations in real time, and in turn, improve the accuracy of the candidate distances for determining the selected entity.

In another embodiment of the present invention, the method further comprises the steps of obtaining cattle data by means of a sensor, processing the cattle data, and determining the task information. Preferably, the cattle data is obtained from the sensors in real time.

With said embodiment, the sensors provide relevant cattle data, which is effective in determining the conditions of cattle. Once the data is processed, the conditions can be established. Then, the task information may be determined preferably correspondingly to the conditions.

Moreover, since the cattle data is obtained in real time, the sensors can provide accurate and up-to-date cattle data. Therefore, the conditions and the task information can be determined accurately.

In another embodiment, the cattle data is measured by the sensor at each predetermined period.

From the embodiments of the invention, it is possible that the cattle data is obtained at the same predetermined period and the conditions can be established at each period. Accordingly, the task information can be determined in a timely manner. Specifically, the communication generated from the sensors may also occur wirelessly, and thus, allowing the conditions and the task information to be determined instantaneously at each period.

In a preferred embodiment, the cattle data includes an acceleration and an angular velocity, wherein the step of processing the cattle data comprises the steps of calculating a plurality of variances of the acceleration in a timeframe and calculating a vector product of the variances.

More preferably, the step of processing the cattle data further comprises the step of calculating a summation of the variances.

Moreover, the method may further comprises the processing the environmental data for determining the task information.

From the above embodiments, the cattle data can be utilized to calculate parameters for determining the conditions of cattle and the task information. The calculated parameters may be compared to a database or a classification in order to evaluate and assign specific conditions of cattle with accuracy, and in turn, determining the accurate task information.

Furthermore, the cattle data can be compared to historical records of specific cattle or specific criteria of data.

Preferably, the above-mentioned steps are carried out by using an artificial intelligence (AI).

As such, the accuracy of the task information may be improved further.

According to another embodiment of the present invention, the task information is used to initiate the step of generating the query for the task. Therefore, the method can respond to the query with the selected entity once there is any task information automatically and in a timely manner.

According to yet another embodiment, the method further comprises a step of generating a notification based upon the task information.

Thus, it is possible to notify a user of any task information that requires user's attention.

Particularly, the user includes owners of the cattle, the candidate entities, the selected entities, and any relevant users. As such, the user can then act appropriately based on the task information and the conditions, regardless of whether it is determined by the method.

In addition to the task information, the aspects pertaining to the candidate entities can be explored in various embodiments of the present invention.

In one embodiment of the present invention, the candidate entities further include a candidate frequency, wherein the candidate frequency are applied the weight factors and calculated for the score.

Additionally, in another embodiment, the candidate entities further include a candidate skill assessment corresponding to the task information, wherein the candidate skill assessment is applied the weight factors and calculated for the score.

From the above embodiments, the candidate frequency can contribute in calculating the score and determining the selected entity that is possibly familiar with the cattle or the cattle owner. Likewise, the candidate skill assessment facilitates in calculating the score and determining the selected entity that is suitable with the task information, particularly tasks required for the cattle.

The aspects of the environmental data can also be provided with more details in various embodiments.

According to an embodiment of the present invention, the environmental data includes at least one indicator selected from a group of temperature, humidity, wind speed, precipitation, and temperature-humidity index (THI).

The environmental data, which provides contextual data relating to the location of cattle, is valuable for evaluating the conditions, determining the corresponding task information, and providing appropriate care.

As a result, the environmental data may be utilized in determining the task information, and can be informed to the user and/or the selected entity in order to provide useful information allowing them to take appropriate actions.

Moreover, according to another embodiment, the environmental data includes an outbreak information for indicating possible outbreak of a disease. Particularly, the outbreak information is applied the weight factors and calculated for the score.

Accordingly, the selected entity can be determined corresponding to potential outbreak in case there are needs for specialized care.

In one embodiment, a risk rating can be assessed using the outbreak information, the task location, and at least one of the temperature, the humidity, the precipitation, the wind speed, and the temperature-humidity index.

Specifically, the risk rating is applied the weight factors and calculated for the score.

Said embodiments would provide the entity or the cattle owner with the risk rating for further evaluation as well as contributing in matching the task with the appropriate entity similarly to other determinants used to calculate the score.

In a specific embodiment, the outbreak information can be added in connection with the task details. As such, the cattle and/or the location can be marked for a potential outbreak and preventive measures can be taken in response to possible risk of spreading.

In the present invention, there is provided a system for matching a task relating to cattle with an entity, comprising: a memory for storing task details including a task information and a task location, a plurality of candidate including a candidate location and a candidate rating, an environmental data corresponding to the task details; and a processor, communicated with the memory and adapted to generate a query for the task relating to cattle, determine at least one selected entity by applying at least one weight factor to the task details, the candidate entities, and the environmental data and calculating a score using the task details, the candidate entities, and the environmental data, and response to the query with the selected entity.

Accordingly, the system is able to obtain relevant information and process the information in order to match the task with the entity appropriately.

Particularly, the processor is further adapted to obtain the task details, the candidate entities, and the environmental data simultaneously upon generating the query for the task relating to cattle. As such, the processor is adapted to obtain the task details, the candidate entities, and the environmental data in real time.

Especially, the processor is further adapted to determine a candidate distance based upon the task location and the candidate location, apply the weight factors to the task information, the candidate distance, the candidate rating, and the environmental data, calculate the score, and determine the selected entity.

Preferably, the process executes its task utilizing an artificial intelligence.

In one embodiment of the present invention, it is provided a sensor adapted to obtain a cattle data and transmit the cattle data to the memory to be obtained by the processor for determining the task information.

As such, information and/or data relating to behavior of the cattle can be obtained from cattle and transferred to other components, particularly the processor, in order to determine an appropriate condition of the cattle as well as the task information, which may be required for the cattle.

Preferably, the sensor is further adapted to measure an acceleration and an angular velocity. As a result, the processor can determine the condition and the task information with accuracy.

In a specific embodiment, the sensor comprises an internal memory wherein the internal memory is adapted to store the cattle data locally. More specifically, the sensor is further adapted to measure the cattle data at each predetermined period.

Therefore, it is possible that a set of data can be created and stored in the internal memory corresponding to the sensor. The set of data is thus exportable, particularly when prompted by the processor.

In another specific embodiment, the processor is further adapted to generate a notification based upon the task information and deliver the notification to a user interface device.

Since the notification is delivered to the user interface device, a user with access to the device can readily be informed of any task information and/or condition that requires their attention. Then, users can response to the task information and/or condition timely.

According to an embodiment of the present invention, the outbreak information is obtained from an external database.

As such, the outbreak information may be retrieved from external source and the coverage of the outbreak information is increased. Furthermore, it is also possible to provide a system and/or a method for extracting the outbreak information from database.

In an alternative embodiment, the outbreak information is added by a user interface device. Hence, it is possible for a user to update the outbreak information manually based on the cattle on site and timely report any possible outbreak.

Various aspects of the present invention include an application of computer-related methods, components, systems, and the likes so as to execute the above embodiments as described. Said application can be conducted wholly or partially depending on needs or requirements.

An embodiment of the invention includes a server for matching a task relating to cattle with an entity utilizing the task details, the candidate entities, and the environmental data according to the above methods.

The present invention also provides a non-transitory computer-readable medium storing instructions which, when executed by one or more processors, cause the processor to carry out the methods provided herein.

The present invention further provides an apparatus including at least one computing device and a computer-readable storage device connected to the computing device and having instructions stored thereon which, when executed by the computing device, cause the computing device to carry out the methods provided herein.

Thus, said apparatus is adapted to implement the methods in accordance with the various embodiments as disclosed.

In one embodiment, the present invention provides a computer executable program comprising instructions which, when the program is executed by a computer, cause the computer to carry out the methods provided herein.

In another embodiment, the present invention provides a computer executable program comprising instructions adapted to perform the methods provided herein.

In yet another embodiment, the present invention also provides a non-transitory computer-readable medium storing a computer executable program comprising instructions for performing operations according to the method provided herein.

### BRIEF DESCRIPTION OF DRAWINGS

The principles of the present invention and their advantages will become apparent in the following description taking into consideration the accompanying drawings in which:

FIG. 1 is a flowchart of a method for matching a task relating to cattle with an entity according to an embodiment of the present invention;

FIG. 2 is a flowchart of the steps to determine the task information according to an embodiment of the present invention;

FIG. 3 is a flowchart of the steps to determine the selected entities according to an embodiment of the present invention; and

FIG. 4 is a schematic diagram of a system for matching a task relating to cattle with an entity according to an embodiment of the present invention.

### DETAILED DESCRIPTION OF EMBODIMENTS OF THE INVENTION

Unless indicated otherwise, certain terminologies are used in the following description for general illustration purposes only and shall not be construed to limit the scope of the concept of the present invention in any way. Likewise, any specific configurations, figures and/or dimensions herein are for illustration purposes and should not be construed to limit the scope of the concept of this technical disclosure.

Unless indicated otherwise, the terminology “cattle” will be inclusive of any bovines that are raised mainly as livestock, irrespective of utility or product they provide, either domesticated or wild. Examples of the meaning will include dairy cattle, beef cattle, cows, bulls, oxen, buffaloes, calves, yaks, and bison, among others which may be appreciated by a person having ordinary skills in the relevant technical field. In addition, the term in the present application encompasses singular meaning. That is to say, it may include an individual, such as one head of cattle, as well as a group of cattle.

The term “entity” used herein will be inclusive of any units, groups of units, persons, individuals, teams, companies, bodies, and the likes being independent from other entities. Specifically, the term may refer to providers, care providers, animal care providers, inseminators, breeders, handlers, veterinarians, technicians, riders, authorities and others persons which may be appreciated in the art.

It must be noted that as used herein and in the appended claims, the singular forms “a”, “an”, and “the” include plural referents unless the context clearly dictates otherwise. Thus, for example, reference to “a component” includes a plurality of components.

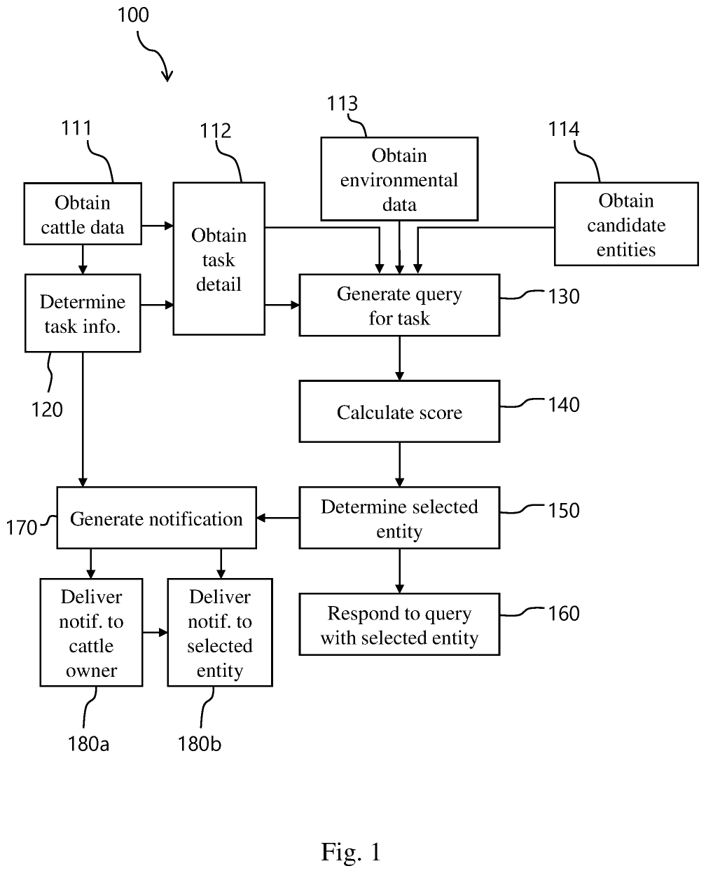

Hereinafter, an embodiment of the present invention will be described in detail with reference to FIGS. 1-4.

FIG. 1 is a flowchart of a method 100 for matching a task relating to cattle with an entity according to an embodiment of the present invention. According to the embodiment, the method 100 comprises the step of obtaining the cattle data 111 by means of sensors, which will be described in detail below. The cattle data includes acceleration and angular velocity. Preferably, the acceleration and the angular velocity include the values in three dimension, specifically in X-axis, Y-axis, and Z-axis. Moreover, the cattle data also include an identification number, date, time, and temperature and the data may be linked to each of the sensors. Preferably, the cattle data is measured by the sensors at each predetermined period from the intervals of 10 seconds to 30 minutes. The cattle data is then transferred and processed in order to perform the step of determining a task information 120. Similarly, the cattle data is transferred at intervals of 5 minutes up to every 30 minutes.

In one embodiment of the present invention, a condition of the cattle is determined initially by using the cattle data and the task information is then determined corresponding to the condition, particularly when the condition is deemed to require a task. Examples of notable conditions are estrus, heat, sickness, parturition, and rest. Correspondingly, certain examples of the task information are insemination, artificial insemination, treatment, and birth delivery.

Then, the step of obtaining task details 112 is performed. The task details at the minimum would include the task information and a task location retrieved based on the cattle, the identification number, user input, and/or an existing database. The task location can simply be registered previously. The task information can also be manually input by a user and a task requirement may be added based on user preference, such as favorite entities and/or preferred product for the task. The task detail including the task information may prompt and initiate the step of generating a query 130 for the task relating to the cattle accordingly.

In the meantime, the steps of obtaining an environmental data 113 and obtaining candidate entities 114 are carried out according to the task details. Particularly, the environmental data is retrieved particularly using the task location while the candidate entities are obtained using the task information. The information on the candidate entities include at least a candidate location and a candidate rating. Optionally, the step of obtaining candidate entities 114 utilizes prerequisites as filters. Said prerequisites are selected manually and/or generated using the task information.

Preferably, the steps of obtaining the task details 112, obtaining the environmental data 113, and obtaining the candidate entities 114, and are conducted simultaneously upon the step of generating the query 130 for the task relating to the cattle. In other words, the task details, the environmental data, and the candidate entities are obtained in real time.

The current environmental data and candidate entities may be obtained upon request. Alternatively, the latest or upcoming environmental data and candidate entities can also be used.

In another embodiment, once the generation of the query 130 is triggered, it is possible that the current cattle data, task details, environmental data, and/or candidate entities will be obtained. Further, the latest or upcoming cattle data, task details, environmental data, and candidate entities may be obtained.

As shown in FIG. 1, the step of calculating scores 140 is further conducted where each score corresponds to each candidate entity. Primarily, the task details, the candidate entities, and the environmental data are applied with at least one weight factor and calculated in order to realize the scores. The weight factors are preferably different based on the type of the information where the factors are configured to prioritize the importance of each information. The step of determining the selected entity 150 is then performed based on the scores where it can be multiple the selected entities. Based on the selected entities, it is provided the step of responding to the query with said selected entity 160.

Moreover, it is also provided the step of generating a notification 170 where the notification can be generated upon determining the task information 120. As an alternative, the notification is generated after determining the selected entities 150 to provide the task information together with the selected entities. The step of delivering the notification to a cattle owner 180a is then carried out to inform the owner of the task information requiring their attention. The owner may choose one or more of the selected entities presented to them and which results in the step of delivering the notification to the selected entities 180b. As also shown, the notification can be delivered to the selected entities directly.

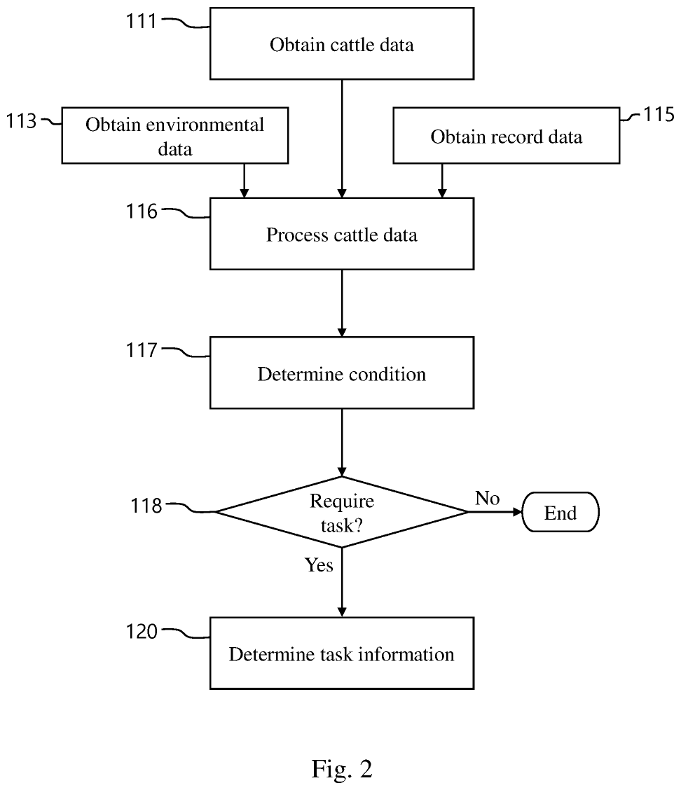

FIG. 2 is a flowchart showing the steps from obtaining of the cattle 111 leading to determining the task information 130 according to an embodiment of the invention. As shown, the cattle data is initially obtained and transferred as described in order to process the cattle data 116, which will be described in detail below.

In processing the cattle data 116, according to the preferred embodiment, the method comprises the steps of calculating variances of the acceleration, which is an absolute difference of the acceleration and the averages of acceleration in a predetermined window or timeframe, resulting in a Dynamic Body Acceleration (DBA). The timeframe are 10, 30, 60, and 120 minutes. Then, Overall Dynamic Body Acceleration (ODBA) is calculated which is a summation of the variances of the acceleration. Particularly, it is determined using the Dynamic Body Acceleration in three axes, which as DBAx, DBAy, and DBAz. Finally, a Vectoral Dynamic Body Acceleration (VeDBA), which is a vector product of the variances, is calculated from a square root of the summations of the absolutes of DBA² in three axes, i.e., DBAx², DBAy², and DBAz².

The calculated values are used as parameters for determining the conditions of cattle 177. Preferably, the parameters are compared to related parameters in a database.

As shown in FIG. 2, the steps include obtaining record data 115 of the cattle. Preferably, historical records of the cattle including records of estrus, sickness, treatment, medication, cattle rating, and history data as well as its behavior in each state are retrieved. As described below, the record data can be included in processing the cattle data 116 as well as determining the condition 117.

In one embodiment, the parameters are compared among various conditions and/or behaviors of the cattle. As an example, the acceleration is compared between estrus and rest conditions of the specific cattle to predict whether the cattle is in heat. The parameters can also be compared to a classification created from the obtained data.

For example, the parameters include behavior of cattle, which can be a simple routine, such as, standing, sleeping, walking, eating, and drinking. The behavior of the cattle is analyzed by measuring its respective duration, such as in hours, minutes, and seconds, and compared to relevant data. As another example, the duration which the cattle spent standing or sleeping is analyzed and compared to the durations indicated as benchmarks in resting and estrus statuses to perform the step of determining the conditions 117. Another means for the analysis is by detecting occurrences of a specific behavior of the cattle in each day, which can be indicative of its health, and performing analysis similarly to the above description.

Preferably, the steps includes obtaining the environmental data 113, where the environmental is used in processing the cattle data 116 and determining the conditions of the cattle 117 in order to improve accuracy. The environment data, including temperature, humidity, wind speed, and precipitation, is measured and obtained similarly to the step of obtaining the cattle data 111. Moreover, a temperature-humidity index may also be calculated and used.

More preferably, the environment data, the processed cattle data, and the record data are applied with weight values and calculated for scores in order to determine the condition 117.

Furthermore, additional data may be acquired to analyze the behavior of the cattle. Said data is optionally acquired from external sources.

Based on the volume and types of data, an artificial intelligence is preferably utilized for determining the condition of the cattle 117.

From the predicted condition, the step of determining the task information 120 is then carried out correspondingly. Specifically, the step of determining necessity of tasks 118 is carried out before determining the task information 120. For example, the condition of the cattle is determined to be in heat and thus the task information will be determined as insemination accordingly. Likewise, if the condition is sickness, then the task information will be treatment. Alternatively, it is possible that the condition will be recognized as normal which does not necessarily require any attention or tasks. In this case, the step of determining the task information 120 is not performed.

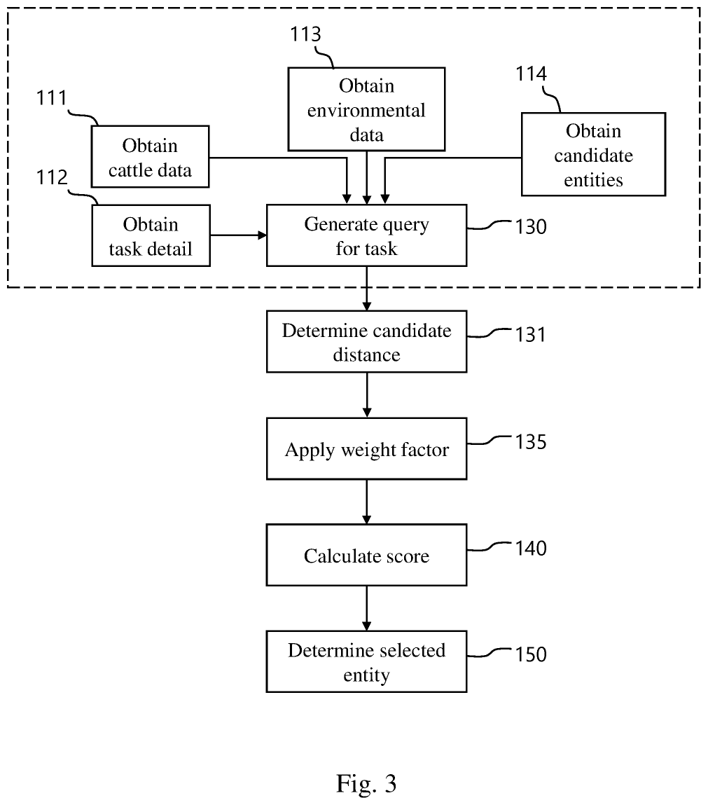

FIG. 3 is a flowchart of the steps leading up to determine the selected entities 150 according to an embodiment of the present invention. In the embodiment, the steps of obtaining the task details 112, obtaining the environmental data 113, and obtaining the candidate entities 114 are conducted relatively simultaneously. Moreover, the step of obtaining the cattle data 111 can also be carried out during this simultaneous timeframe.

As shown in FIG. 3, the steps of obtaining the cattle data 111, obtaining the task details 112, obtaining the environmental data 113, and obtaining the candidate entities 114 are conducted relatively simultaneously upon generating the query for the task relating to cattle 130. In a practical manner, the mentioned steps in obtaining data 111, 112, 113, 114 utilize each latest data available.

According to an embodiment of the invention, each task detail includes a task information and a task location and each candidate entity includes at least a candidate location and a candidate rating. Various embodiments on the candidate location and the candidate rating are described in more detail below.

In the preferred embodiment, the candidate rating includes an overall rating and a personal rating of the entity associated to a user (e.g. a cattle owner). It may also include recorded reports of history related to the entity. As a result, the user will be able to match with the preferred entity. The candidate rating is preferably based on past records connected to the entity. Generally, a user or a cattle owner will be able to provide ratings for the entity after receiving their services in order to indicate their satisfaction as well as potentially provide a skill score for the tasks. Preferably, the candidate rating is adjusted dynamically at the same time upon receipt of ratings.

In obtaining the candidate entities 114, the candidate location is preferably obtained from records containing the candidate location generated upon an interaction of the candidate entity at the beginning of each day. Alternatively, the candidate location is acquired in real time upon specific requests.

In another embodiment of the invention, the task location and the candidate location are processed to determine a candidate distance 131.

Additionally, in determining the candidate distance 131, the calculation of the candidate location and the task location can take into account traffic information of possible routes. Alternatively, it is possible that the candidate distance is used as a filter for limiting eligible candidate entities in an area.

The task information, the candidate distance, the candidate rating, and the environmental data are then subjected with the step of applying weight factors 135.

As described, the weight factors are preferably vary based on the type of the information and its importance. The weight factors can be determined by various data, including data generated by machine learning, recorded data, and manual input.

In one embodiment, each candidate entity further includes a candidate skill assessment corresponding to the task information. Similarly, the candidate frequency is also subjected with the step of applying weight factors 135. Preferably, the candidate skill assessment is determined specific to the task information, that is, the skill assessment takes into account an expertise of the entity in specific tasks. For example, the skill assessment or a skill scoring is calculated based on trainings and certifications the entity has received. The skill assessment can be connected to roles and responsibilities of the entity and it may further include their accuracy in performing services. Further, the candidate skill assessment can be related to the candidate rating of the candidate entity. Specifically, the skill assessment includes equipment available to the entities.

In another embodiment, each candidate entity further includes a candidate frequency. The candidate frequency includes the number of times the entity has provided their service a cattle and/or a user such as a cattle owner. As a result, it is possible to highlight the selected entities who have provided frequent services to the user for their consideration. Similarly, the candidate frequency is also subjected with the step of applying weight factors 135.

Various embodiments on the environmental data from the step of obtaining the environmental data 113 are also described below.

As mentioned, the environmental data, which is subjected with the step of applying weight factors 135, includes at least one of temperature, humidity, wind speed, precipitation, and calculated temperature-humidity index.

Preferably, parts of the environmental data are retrieved from external source. For example, the wind speed is obtained using on site measurement and weather information of the location is acquired from external sources. With both information, it is possible to forecast future weather in the area of the location.

In one embodiment of the invention, the environmental data includes an outbreak information for indicating possible outbreak of disease. Likewise, the outbreak information is preferably subjected with the step of applying weight factors 135. The outbreak information can be obtained from internal records in connection with a management system for checking the cattle possibly at risk of causing outbreak. In addition, the outbreak information can also be retrieved from external source. For example, it is possible to look out for events and occurrences of any outbreaks in the area from social media through analyzing text messages of relevant authority in the area as well as individuals from inside and outside the area. Furthermore, the outbreak information can be manually added in connection with the task details upon detection, and thus, the outbreak information will be connected the task location of the task details.

From the outbreak information, a risk rating can be assessed using in combination with the location and at least one of the temperature, the humidity, the precipitation, and the wind speed, which is preferably obtained by measurement. For example, the risk rating is calculated and categorized into high, medium, and low. Then, the rating is assigned to areas in order to provide visualization of the risk rating. The information may be used to inform relevant users or authorities so that treatments can be provided timely. Similarly, the risk rating is preferably subjected with the step of applying weight factors 135.

Afterward, the step of calculating a score for each candidate entity 140 is performed based on above-mentioned information that have been obtained and subjected to the step of applying with the weight factors 135. The score of each candidate entity are sorted and compared in order to determine the selected entities 150. As a result, the selected entities which are ideally the most suitable candidates for performing the task can be further provided to users.

In determining the selected entity 150, it is possible to determine multiple entities where a user or the cattle owner will be able to make a further selection, preferably after a notification is sent to a cattle owner.

As an example of various embodiments, the notification is sent informing the cattle owner of estrus detection in cattle and a list of the selected entities is displayed along with the respective rating. The selected entities are specialized in insemination located within the area of the task location. Additional information on the task is also provided including date and time, identification number of the cattle, symptoms, the determined condition, severity scoring, and brief historical records of the cattle. The cattle owner then chooses one of the selected entities to perform the insemination and the notification is sent to the chosen entity advising them of the task details, the cattle, and the location. Once the chosen entity confirms the task, a listed of equipment is generated based on the task and distributed to the cattle owner and the chosen entity for cost estimations and preparation, respectively. The owner can confirm the task accordingly in order to complete the matching process. After the task is performed, as mentioned, the owner may rate the chosen entity in order to be used in further matching. The ratings are recorded and reflected automatically.

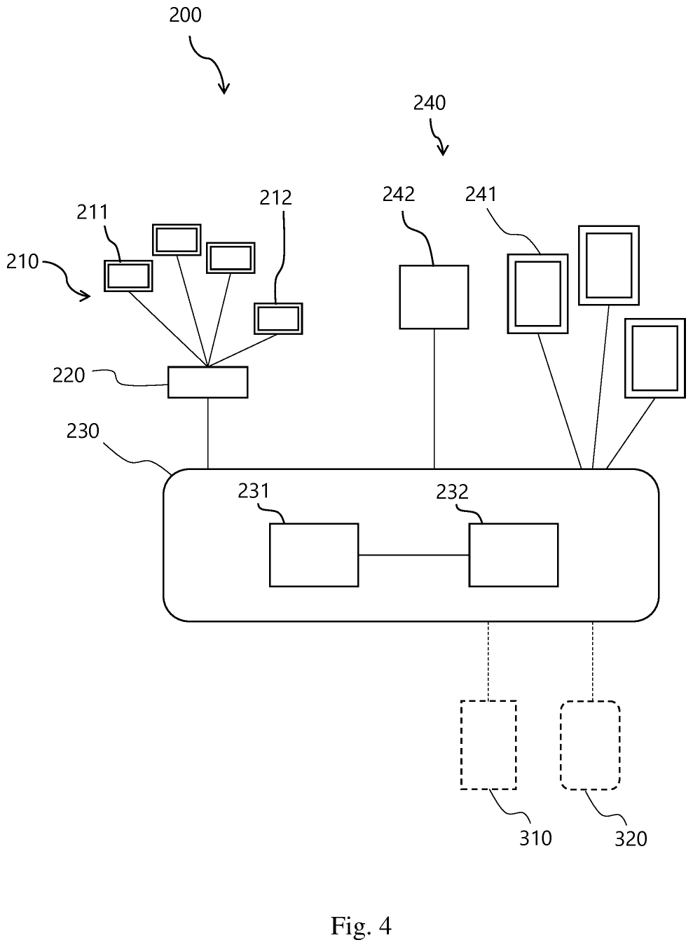

FIG. 4 is a schematic diagram of a system 200 for matching a task relating to cattle with an entity according to an embodiment of the present invention.

According to an embodiment of the invention, sensors 210 are provided to obtain the cattle data and the environmental data. For the cattle data, a cattle sensor 211 is preferably attached near the neck of each individual cattle. Preferably, the sensors 210 are attached to the top-right position of the neck of each cattle. The sensors 210 include a gyroscope adapted to measure angular velocity, specifically the change in angular velocity, in the ranges of 250, 500, 1000, 2000 degree/s and an accelerometer adapted to measure the acceleration in the ranges of ±2, ±4, ±8, ±16 g. Preferably, the sensors 210 measure the data at the interval of 10 seconds. As for the environmental data, some of the sensors 210 are adapted to measure temperature and humidity at the cattle location such that it is possible to calculate for a temperature-humidity index. Specifically, an environment sensor 212 is provided on cattle field for obtaining specifically parts of the environmental data at an interval. More preferably, the environment sensor 212 is also configured to measure wind speed and precipitation.

Moreover, the sensors 210 further comprises a gateway for transmitting low-frequency signal to a local network 220 which then transmit data further to a server 230. The gateway transmits the signal at an interval, preferably every 30 minutes.

In the preferred embodiment, the sensor 210 comprises an internal memory for storing the cattle data allowing the sensor 210 to measure the cattle data at each predetermined period, such as every 10 seconds, and record a set of data in the internal memory. The internal memory is preferred due to the transmission of the gateway, which is performed at lower rate than that of the sensors 210.

According to an embodiment of the present invention, the server 230 comprises a memory 231 and a processor 232 communicated with the memory 231. The processor 232 is configured to determining the condition of the cattle and the task information accordingly to the above embodiments using the data measured by the sensors 210, preferably stored in the memory 231. The condition and the task information may then be stored in the memory 231.

In addition, the processor 232 is configured to generate a query for the task relating to cattle. Further, the processor 232 is also configured to obtain task details, candidate entities, and environmental data simultaneously upon generating the query for the task. The processor 232 may obtain the task detail, the candidate entities, and the environmental data from the memory 231 or through a gateway adapted to communicate with other components, such as the local network 220 which in turn communicate to the sensors 210. The processor 232 is further configured to apply weight factors to the task details, the candidate entities, and the environmental data and calculate a score for each entity. The weight factors may be generated by the processor 232 or obtained from the memory 231.

The memory 231 is configured to store the task detail including a task information and a task location, the candidate entities including candidate locations and candidate ratings, the environmental data, including the outbreak information. In the preferred embodiment, the memory 231 is further configured to additional data, which can be utilized to determine the conditions of the cattle as described. The additional data includes historical records of the cattle, cattle movement data, cattle identification data, time of each store movement, age, identification number, location of latest notable event for provided to the processor 232 to determine the condition and the task information.

In an embodiment, the candidate entities, particularly the candidate locations, are obtained from a user interface device 240, specifically, an entity device 241 and then stored in the memory 231. In a specific embodiment, the processor 232 is configured to calculate a candidate distance using the stored candidate location in the memory 231.

Additionally, the processor 232 is configured to respond to the query with the selected entities.

Preferably, the server 230 utilizes an artificial intelligence for determining the condition, the task information, and the selected entities.

In the preferred embodiment, an application software is provided for communication between the server 230 and the user interface device 240. For example, the processor 232 generates a notification of the task information and the selected entities and delivers the notification to the user interface device 240, specifically the cattle owner device 242, by means of the application software. Similarly, a notification of the task information can also be delivered to the entity device 241 of the selected entity chosen by the cattle owner.

As another example of various embodiments, the notification and a list of the selected entities is sent and displayed using the application software in the cattle owner device 242. The cattle owner then chooses one of the selected entities to perform the task in the software on their device 242. Then, a notification is sent to the chosen entity also informing them of the task on the entity device 241 via the application software. Other communications among the cattle owner, the chosen entity, the server 230, and the devices 240 can be performed using the software accordingly.

Furthermore, the entity device 241 may communicate the outbreak information to the server 230 if required.

In an embodiment, an external database 310 is utilized for retrieving additional information, including the outbreak information, location information, and weather information.

Additionally, the server 230 may detect any outbreak information from an external system 320.

While this invention has been described in conjunction with the examples of embodiments outlined above, various alternatives, modifications, variations, improvements and/or substantial equivalents, whether known or that are or may be presently foreseen, may become apparent to those having at least ordinary skills in the art. Accordingly, the examples of embodiments of the invention, as set forth above, are intended to be illustrative, not limiting. Various changes may be made without departing from the spirit or scope of the invention. Therefore, the invention is intended to embrace all known or earlier developed alternatives, modifications, variations, improvements and/or substantial equivalents. The present invention may include any combination of the aspects and features described herein. That is, the invention also includes, but is not limited to, the combinations of aspects and features disclosed herein.

1. A method for matching a task relating to cattle with an entity, comprising the steps of:
generating a query for the task relating to cattle;
obtaining task details, wherein the task details include a task information and a task location;
obtaining a plurality of candidate entities using the task information, wherein the candidate entities include a candidate location and a candidate rating;
obtaining environmental data corresponding to the task details;
determining at least one selected entity by applying at least one weight factor to the task details, the candidate entities, and the environmental data and calculating a score using the task details, the candidate entities, and the environmental data; and
responding to the query with the selected entity.

2. The method of claim 1, wherein obtaining the task details, obtaining the candidate entities, and obtaining the environmental data are conducted simultaneously upon generating the query for the task relating to cattle.

3. The method of claim 1, wherein the task location and the candidate location are processed to determine a candidate distance, and wherein the task information, the candidate distance, the candidate rating, and the environmental data are applied the weight factors and calculated for the score to determine the selected entity.

4. The method of claim 1, further comprising
obtaining cattle data using a sensor,
processing the cattle data, and
determining the task information.

5. The method of claim 4, wherein the cattle data includes an acceleration and an angular velocity, wherein processing the cattle data comprises calculating a plurality of variances of the acceleration in a timeframe and calculating a vector product of the variances.

6. The method of claim 5, wherein processing the cattle data further comprises calculating a summation of the variances.

7. The method of claim 4, wherein the cattle data is measured by the sensor at each predetermined period.

8. The method of claim 4, wherein the task information is used to initiate generating the query for the task.

9. The method of claim 1, further comprising generating a notification based upon the task information.

10. The method of claim 1, wherein the candidate entities further include a candidate frequency, the candidate frequency being applied the weight factors and calculated for the score.

11. The method of claim 1, wherein the candidate entities further include a candidate skill assessment corresponding to the task information, the candidate skill assessment being applied the weight factors and calculated for the score.

12. The method of claim 1, wherein the environmental data includes at least one of temperature, humidity, wind speed, precipitation, and temperature-humidity index.

13. The method of claim 1, wherein the environmental data includes an outbreak information for indicating possible outbreak of a disease.

14. The method of claim 13, wherein the outbreak information is applied the weight factors and calculated for the score.

15. The method of claim 13, wherein a risk rating can be assessed using the outbreak information, the task location, and at least one of the temperature, the humidity, the precipitation, the wind speed, and the temperature-humidity index.

16. The method of claim 15, wherein the risk rating is applied the weight factors and calculated for the score.

17. The method of claim 13, wherein the outbreak information can be added in connection with the task details.

18. A system for matching a task relating to cattle with an entity, comprising:
a memory for storing task details including a task information and a task location, a plurality of candidates including a candidate location and a candidate rating, an environmental data corresponding to the task details; and
a processor, communicated with the memory and configured to
generate a query for the task relating to cattle,
determine at least one selected entity by applying at least one weight factor to the task details, the candidate entities, and the environmental data and calculating a score using the task details, the candidate entities, and the environmental data, and
respond to the query with the selected entity.

19. The system of claim 18, wherein the processor is further configured to obtain the task details, the candidate entities, and the environmental data simultaneously upon generating the query for the task relating to cattle.

20. The system of claim 18, wherein the processor is further configured to
determine a candidate distance based upon the task location and the candidate location,
apply the weight factors to the task information, the candidate distance, the candidate rating, and the environmental data,
calculate the score, and
determine the selected entity.

21. The system of claim 18, further comprising a sensor configured to obtain cattle data and transmit the cattle data to the memory to be obtained by the processor for determining the task information.

22. The system of claim 21, wherein the sensor is further configured to measure an acceleration and an angular velocity.

23. The system of claim 21, wherein the sensor comprises an internal memory, the internal memory being configured to store the cattle data locally, and wherein the sensor is further configured to measure the cattle data at each predetermined period.

24. The system of claim 18, wherein the processor is further configured to generate a notification based upon the task information and deliver the notification to a user interface device.

25. The system of claim 18, wherein the environmental data includes outbreak information for indicating possible outbreak of a disease, the outbreak information being obtained from an external database.

26. The system of claim 18, wherein the environmental data includes outbreak information for indicating possible outbreak of a disease, the outbreak information being added by a user interface device.

27. A non-transitory computer-readable medium storing instructions which, when executed by one or more processors, cause the one or more processors to carry out the method of claim 1.

28. An apparatus, including:
a computing device; and
a non-transitory computer-readable storage device connected to the computing device and having instructions stored thereon which, when executed by the computing device, cause the computing device to carry out the method of claim 1.

29. (canceled)
